(19) United States
(12) Patent Application Publication
(10) Pub. No.: US 2011/0313055 A1
(43) Pub. Date: Dec. 22, 2011

(54) HEALTH CHARACTERISTIC CHEWY OR GUMMY CANDY CONFECTION

(75) Inventors: Gary Ervin, Naples, FL (US); Erin Ervin, Naples, FL (US)

(73) Assignee: ANTIOXIDANT SUPERFOODS, INC., Naples, FL (US)

(21) Appl. No.: 13/163,197

(22) Filed: Jun. 17, 2011

Related U.S. Application Data
(60) Provisional application No. 61/355,562, filed on Jun. 17, 2010.

(51) Int. Cl. A23G 3/42; A23G 3/48; A23L 1/275; A61P 19/08; A61P 9/00; A61P 3/00; A61P 3/02; A61P 3/06; A23L 1/308; A61K 47/46

(52) U.S. Cl. 514/777; 426/648; 426/573; 426/250; 514/783

(57) ABSTRACT

The present invention provides for flavored chewy or gummy candy confections made by making a flavored chewy or gummy candy confection from scratch or adjusting a current flavored chewy or gummy candy confection brand product with our invention. This invention can have a healthier group of edible oils component, unique antioxidant taste profile component, fiber component, water component, emulsifier component, and potentially a 0% or greater supplement component that can add additional dietary benefits. Specific ratios of ingredients can lower sugar content up to 75% while creating a product with a taste, texture, and mouth feel similar to regular flavored chewy or gummy candy. The reduction of sugar possible within this invention can reduce the negative health ramifications of high sugar content that current flavored chewy or gummy candy confections suffer from and the addition of the healthier ingredients adds some improved health characteristics to the products that usually are devoid of nutrition. Additionally, the method for preparing a flavored chewy or gummy candy confection with these improved health characteristics, taste profile, and dietary benefits are covered.

This application claims priority to provisional application Ser. No. 61/355,562, filed on Jun. 17, 2010, the disclosure of which is incorporated by reference herein in its entirety.

### FIELD OF THE INVENTION

The embodiments relate generally to ingestible flavored chewy or gummy candy confectionery compositions that have improved health characteristics, lower sugar content, higher antioxidant content, balance of dietary fats and beneficial dietary fiber. Supplements, nutraceuticals, or medicines can be added to the flavored chewy or gummy candy confections to target certain health issues such as calcium for healthy bones, cholesterol control or triglyceride control that supports prevention of cardiovascular disease, protein supplementation, vitamins and minerals that support health and other potential health issues.

### DESCRIPTION OF RELATED ART

Chewy flavored candy or gummy confections currently available in the marketplace are produced in one of four ways. First, many flavored chewy or gummy confections are made fat free utilizing monosaccharides and disaccharides as the majority of the ingredients. These fat free flavored chewy or gummy candy confections exhibit high sugar levels in the range of 50% plus sugar, have little nutritional value, and contribute to the health problems that high sugar content in our diets is creating including overweight, obesity, type II diabetes, and cardiovascular disease.

Second, many flavored chewy or gummy confections currently available in the market place are made with partially or fully hydrogenated oils and high levels of monosaccharides and disaccharides as the majority of the ingredients. These flavored chewy or gummy confections use forms of oils that have Trans Fat within the oils and partially and fully hydrogenated oil consumption has been linked to increased risk of developing cardiovascular and heart disease. These flavored chewy confections have richer taste, texture, and mouth feel characteristics compared to fat free flavored chewy or gummy candy confections. The flavored chewy or gummy candy confections made with partially or fully hydrogenated oils also have high sugar contents usually in excess of 50% plus sugar, have little nutritional value, and contribute to the health problems that high sugar content in our diets are creating as well as contribute unhealthy oils to the diet.

Third are the types of flavored chewy or gummy confections that use fats such as palm kernel oil, palm oil or coconut oils, alone or in combinations. The flavored chewy or gummy candy confections that utilize these oils are superior from a health perspective vs. those that use partially and fully hydrogenated oils because these oils don't have Trans fat as part of their structure. These types of chewy or gummy candy confections still have high sugar contents usually in excess of 50% plus sugar. The edible oils within these types of flavored chewy candy confections do have some health benefits, but the high sugar content within the products outweighs any positive benefits from the edible oils.

Fourth, are flavored chewy or gummy candy confections that have the carbohydrate sweeteners either partially or completely replaced by polyols or sugar alcohols. The flavored chewy or gummy confections can either be fat free, have partially or fully hydrogenated oils in the compositions or can have palm kernel oil, palm oil or coconut oils alone or in combination. Polyols or sugar alcohol essentially makes the flavored chewy or gummy confection sugar free. These confections tend to not taste as good as flavored chewy or gummy confections made with regular sugar based sweeteners.

U.S. Pat. No. 6,663,910 to Soumya, et al., describes the preparation of food products with carrageen that have a carbohydrate sweetener component and a gelling component. This patent is said to facilitate a high solids level while being pourable at a higher temperature. This product also is said to have a moisture content at 13% to 25%. The products of Soumya, et al., are essentially a flavored chewy or gummy candy confection.

U.S. Pat. No 5,476,678 to Walter, et al., describes the preparation of fiber fortified chewy confection type candies. Walter, et al. includes a dry mixture of ingredients selected from maltodextrin, fondant sugar, powdered sugar, cream flavor, organic food acids, and yogurt powder and mixtures thereof, an emulsifier, a syrup system, a forticant, and a dietary fiber component. Walter, et al. does not appear to incorporate an edible oil component with highly unsaturated and highly saturated content, a vanilla powder component, or fibers such as inulin, fructooligosaccharides, or wheat dextrin as the preferred fiber component. Walter, et. al. does not appear to include a liquid component as a primary ingredient within its composition or any edible oil component at all.

U.S. Patent Application Publication No 20100112142 to Barkalow, David G, et al. filed February 2008 describes a flavored chewy confectionery product comprising erythritol and methods of producing the confectionery products. Barkalow, et al. use erythritol as the main suggested nutritive sweetener in place of monosaccharides and disaccharides. Barkalow, et al. also use a fat selected from the group consisting of palm kernel oil, hydrogenated palm kernel oil, cocoa butter, fractionated palm kernel oil, hydrogenated and partially hydrogenated vegetable oil and combinations thereof.

U.S. Patent Application Publication No 20090123597 to Williams, Kristin Rhedrick, et al.; filed Nov. 7, 2007 describes a flavored chewy confection type product that can be used as a dietary fiber supplement that delivers a therapeutic amount of fiber in a small chew or other structure. Williams, et al. incorporate a fiber component at a minimum of 25%, and in most of the invention embodiments the structure is not a confection, but rather a delivery method for dietary fiber utilizing only inulin and a delivery platform. Williams, et. al. make no mention of using its invention to adjust or improve the health characteristics of any other confection, and utilizes partially hydrogenated coconut and milk fat as the preferred fats, which are unhealthy fats.

In the 1980's the FDA established food claims guidelines including ones that were based on a “low fat” diet being important to reduce the risk of coronary heart disease and to improve consumer's health. Through continued scientific study it has been found that it is not the amount of fat in the diet per se that is the critical aspect for reducing health related issues like; coronary heart disease, diabetes, obesity, and other health issues, but rather the types of fats and the balance of those fats that are included in foods. (see http://www.hsph.harvard.edu/nutritionsource/what-should-you-eat/fats-full-story/index.html)

As food producers responded to the FDA guidelines of the 1980s many “low fat” foods including confectionery foods like Welch's Fruit Snacks fat free, Red Vines fat free, and Twizzlers low fat flavored chewy or gummy candy brands were introduced. Many of the flavored chewy or gummy candy claim to be fat free or low in fat as if this were a positive health claim. Generally when a flavored chewy or gummy candy is fat free it means its sugar content is just higher. The 2005 USDA Dietary Guidelines, however, now suggest under its fat recommendations that consumers should reduce the amount of saturated fat in the diet and replace it with unsaturated fatty acids like monounsaturated and polyunsaturated fats, and include some saturated fats within the diet preferably from sources like coconut oil that has mostly medium chain triglycerides that produce energy in the body and do not convert to stored fat. Consensus in the medical community, based on clinical research and food studies, is that increased levels of monounsaturated and polyunsaturated fats in the diet would help in the prevention of cardiovascular disease and coronary heart disease related problems.

The link below discusses the Harvard School of Public Health study that was the largest food study ever created to examine how certain fats affect consumer's long term health. The conclusion is that consumers need to get more of the “good unsaturated fats” (unsaturated fats including monounsaturated and polyunsaturated fats) and less of the saturated fats in their diets. The embodiments described herein provide flavored chewy or gummy candy confections that have new structures to improve the taste, texture, melt, and mouth feel while providing beneficial monounsaturated and polyunsaturated fat content. Certain embodiments can add the essential fatty acids Omega 6 and Omega 3 ALA's that are not currently found in flavored chewy or gummy candy confections. Many of the flavored chewy or gummy candy include fully or partially hydrogenated oils including Skittles, Starbursts, Airheads, Tootsie Rolls, etc. and these products contribute Trans fats to the diet that are even more detrimental to health then the saturated fats.

http://www.hsph.harvard.edu/nutritionsource/what-should-you-eat/fats-full-story/index.html

This report is based on the largest study ever undertaken on the effect of reducing saturated fats and replacing them with unsaturated fats including monounsaturated and polyunsaturated fats. The study concluded that many of the major health issues facing consumers today can be directly attributed to consumption of high levels of saturated fat, partially hydrogenated, fully hydrogenated, and Trans fats. The study examined the effect of replacing such saturated fats with healthier monounsaturated and polyunsaturated fats in the diet and how certain saturated fats like those found in coconut are significantly better for you than saturated fat found in milk and butter. The study concluded that this had a significant effect on reducing the risks of developing chronic health conditions including cardiovascular disease and coronary heart disease. Our flavored chewy or gummy candy confections have components that support the research found by the above Harvard study.

The description herein of certain advantages and/or disadvantages of known products or methods is not intended to remove some or all of the components of these known products or methods from the scope of the present embodiments. Indeed, some embodiments described herein may incorporate some or all of the components of the known products or methods, without suffering from the disadvantages. The description provided in this section also is not intended as an admission that the documents or products are prior art, especially the description of the embodiments described herein.

### SUMMARY OF INVENTION

It is a feature of an embodiment of the invention to provide a good taste, texture, melt, and mouth feel flavored chewy or gummy candy confection without the necessity of utilizing partially or fully hydrogenated fats that have negative health consequences. This flavored chewy or gummy candy confection can be made in the typical manner or can be made utilizing an already made flavored chewy or gummy candy confection as the base for the composition. It is another feature of an embodiment of the present invention to create a flavored chewy or gummy candy confection that can have an improved balance of dietary fats including saturated fats, polyunsaturated fats, and monounsaturated fats including beneficial Omega 6 and Omega 3 fatty acids. It is another feature of an embodiment of the invention to establish an antioxidant flavor profile that gives the flavored chewy or gummy candy confection a specific flavor that comes from the synergistic blend of coconut oil, vanilla powder, and the edible oils. The flavor profile thus created is a unique taste profile, when compared to other commercially available flavored chewy or gummy candy confections.

It is another feature of an embodiment of the present invention to create flavored chewy or gummy candy confections that include a beneficial dietary fiber component. This dietary fiber component provides an improved texture and mouth feel as well as positive health attributes. Inulin, fructoligiosaccharides, and wheat dextrin are the preferred fiber sources.

It is another feature of an embodiment of the invention to reduce the sugar content in flavored chewy or gummy candy confections by 25% to 75%, when compared to conventional commercially available flavored chewy or gummy candy confection brands. The present embodiments can lower the sugar content while replacing it with healthy edible oils, an antioxidant flavor profile blend, beneficial dietary fiber, water, and additional supplement ingredients in certain compositions. Eating foods high in sugar is tied directly to negative health issues including being overweight, obesity, type II diabetes, cardiovascular disease, heart disease and other health issues. Many flavored chewy or gummy candy confections also use partially hydrogenated oils or fully hydrogenated oils within their compositions, such as Starbursts, Skittles, Airheads, Tootsie Rolls, and many others. These partially hydrogenated oils or fully hydrogenated oils have Trans fats within them and contribute to negative health issues like cardiovascular disease and heart disease. The present embodiment can create comparable tasting products that replace the partially hydrogenated or fully hydrogenated oils with a healthier group of edible oils. Alternatively, in the case where the health structure of an existing flavored chewy or gummy confection product is improved, the embodiments described herein can add beneficial monounsaturated and polyunsaturated fats to the compositions. Another feature of the embodiments is to remove partially or fully hydrogenated oils from the based flavored chewy or gummy confection that uses these oils, and to and replace them with a healthier group of edible oils.

It is another feature of an embodiment of this invention to reduce the calorie content in flavored chewy or gummy candy confections. The preferred embodiments make use of a dietary fiber component that can reduce the calorie content in the flavored chewy candy confection, depending on the type of dietary fiber utilized. Sugar has a calorie content of around 4.5 calories per gram and fat has a calorie content of around 8.5 calories per gram, compared to fructoligiosaccharides, a dietary fiber that has only 1.8 calories per gram. In certain preferred embodiments, fructooligiosaccharides are utilized as the dietary fiber source, and thus will lower the calorie content of the flavored chewy or gummy candy confection. The preferred embodiments also are capable of significantly reducing the viscosity of the confection, and water can be added as an ingredient into the composition to compensate for the reduction in viscosity. The use of fructooligiosaccharides and water can provide a 20% to 30% reduction in calories, when compared to regular formulas with similar taste profiles. Calories in flavored chewy or gummy candy confections are a problem because many packages can have 600 to over 1000 calories per container, and consumers often eat an entire package in one sitting. Examples of flavored chewy or gummy confections with such high calorie contents include Twizzlers, Starbursts, Skittles, Sour Patch Kids, and most of the Wonka's brand chewy or gummy flavored candies, as well as other major brands sold in the hook display type of bags and multiple serving container boxes. Most of the same flavored chewy or gummy confections sold for Movie snacks also have calorie contents this high. It is a feature of an embodiment to reduce the calorie content of such flavored chewy or gummy confections.

In accordance with these an other features of embodiments, there is provided a composition comprising: a. a monosaccharide component from about 10% to about 40% by weight, based on the total weight of the composition; b. a disaccharide component from about 10% to about 40% by weight, based on the total weight of the composition; and c. a dietary fiber component present in an amount of from about 0.001% to about 80% by weight, based on the total weight of the composition.

In accordance with another feature of an embodiment of the present invention, there is provided a modified flavored chewy or gummy confection candy comprising: a. a flavored chewy or gummy confection candy present in an amount of from about 30% to about 90% by weight, based on the total weight of the composition; b. a fiber component present in an amount of from about 0.001 to about 60% by weight of the composition; and c. a water component from about 1% to 20%, by weight of the composition.

In accordance with another feature of an embodiment of the present invention, there is provided a method of making a chewy or gummy candy confection comprising: (a) mixing at least a monosaccharide component, a disaccharide component, and a dietary fiber component in a vessel; (b) heating the mixture, while mixing and scraping, to a temperature within the range of from about 60 to about 82.2° C. for a period of time of from about 5 minutes to about 30 minutes to provide a homogeneous mixture; (c) optionally cooling the mixture to a temperature of about 37.8° C. and adding a natural preservative; (d) processing the mixture to form a chewy or gummy candy confection.

In accordance with another feature of an embodiment of the present invention, there is provided a method of making a modified chewy or gummy candy confection comprising: (a) mixing at least a chewy or gummy candy confection, a dietary fiber component, and a water component in a vessel; (b) heating the mixture, while mixing and scraping, to a temperature within the range of from about 60 to about 82.2° C. for a period of time of from about 5 minutes to about 30 minutes to provide a homogeneous mixture; (c) optionally cooling the mixture to a temperature of about 37.8° C. and adding a natural preservative; (d) optionally adding additional flavors and colors to match the original flavor and color of the chewy or gummy candy confection added in (a); and (e) processing the mixture to form a chewy or gummy candy confection.

These and other objects and advantages of the present invention shall become apparent from the detailed description that follows.

### DETAILED DESCRIPTION OF THE INVENTION

All percentages and ratios are calculated by weight unless otherwise indicated. All percentages and ratios are calculated based on the total final composition unless otherwise indicated. All numerical values described herein are intended to include experimental measurement error within their value, up to about 10% of the value. For example, reference to an amount of 5% by weight, includes within its scope 4.5% to 5.5% by weight. Referenced herein also may be trade names for components including various ingredients utilized in the present invention. The inventors herein do not intend to be limited by materials under a given trade name. Equivalent materials (e.g., those obtained from a different source under a different name or reference number) to those referenced by trade name may be substituted and utilized in the description.

As used herein “fiber” generally denotes a material derived from plant cell walls and which is not digestible by human digestive enzymes, including soluble fiber and insoluble fiber. The fiber component can be naturally derived or synthetic. A portion of the fiber component can be non-starch polysaccharides, including soluble and non soluble fiber. As used herein, “soluble fiber” denotes plant gums and oligosaccharides, or modified gums, modified celluloses, non-starch polysaccharides that are soluble in water, some of which can form viscous gels.

The preferred embodiments described herein are intended to improve the health characteristics of flavored chewy or gummy confections, and to expand the potential consumer market for such confections by providing equivalent taste, texture, and mouth feel flavored chewy or gummy confections as commercially available flavored chewy or gummy confections. The preferred embodiments also are intended to provide these benefits without the inclusion of ingredients such as partially or fully hydrogenated oils or both which exhibit negative health characteristics. In some embodiments in which a flavored chewy or gummy confection that has partially hydrogenated or fully hydrogenated oil within its composition is used as the base (e.g., the confection is not prepared from scratch, but rather is a modification of an existing chewy or gummy confection), the preferred embodiments can improve the fat structure through the addition of beneficial monounsaturated and polyunsaturated oils as well as Omega 6 and Omega 3 ALA's. It may be possible to remove the partially or fully hydrogenated oils from the existing compositions and replace them with healthier edible oils.

Many producers have created flavored chewy or gummy confections that are claimed to be “fat free” (e.g. Welch's fruit snacks, Swedish Fish, etc.). Consumers purchase such products for their purported heath benefits, without realizing that the “fat free” status of the formulation has been created by substituting sugar for fat, and the consumer is made to think fat free is healthy when the right types of fat actually brings health benefits to a flavored chewy or gummy confection. Sugar is known to be one of the largest contributing factors to health problems and the many healthier alternative edible oils and fats now available as well as dietary fiber can displace this sugar and provide flavored chewy or gummy confections with an improved health characteristic structure. Some of the preferred embodiments displace sugar with alternative healthy edible oils, with dietary fiber, and with water in certain embodiments, thereby reducing the sugar content by as much as 75%, while still providing the consumer with a healthy group of dietary fats and beneficial dietary fiber and a final flavored chewy or gummy candy that has great taste.

While not intending on being bound by any theory of operation, the group of nutritive ingredients utilized in the preferred embodiments is believed to improve the flavor and taste profile of flavored chewy or gummy candy confections, while substantially reducing the sugar levels and the fiber content, and at the same time, causing the carbohydrates to be processed slower similar to how sugar alcohols are processed within the body. It also is believed, again not wishing to be bound by any theory of operation, that the preferred embodiments can provide a flavored chewy or gummy confection that has similar effects on blood sugar as flavored chewy or gummy confections made with sugar alcohols, while maintaining a superior taste profile.

Certain embodiment also provide flavored chewy or gummy candy confection compositions that exhibit great taste, texture, melt, and mouth feel without incorporating any partially or fully hydrogenated oils. The embodiments also can be used to adjust an existing flavored chewy or gummy candy confection so that it has an improved nutritive structure. This improved nutritional structure can have dietary fiber. Other embodiments can adjust an existing flavored chewy or gummy candy confections so that it includes improved health characteristics, antioxidant content, reduction in sugar content, balance of dietary fats and addition of beneficial dietary fiber that can have probiotic benefits and potentially additional nutritive dietary ingredients.

One of the features of the present invention is to improve the health characteristics of flavored chewy or gummy confections while maintaining great taste, texture, melt, and mouth feel of the product, and do so without the inclusion of partially or fully hydrogenated oils that add Trans fat to flavored chewy or gummy confections. Another aspect of this invention is to create flavored chewy or gummy confection compositions that have nutritional contents that can be widely recommended by the medical community as a healthy food choice. Certain preferred combinations of ingredients can provide beneficial dietary fiber, significantly lower sugar content, add essential fats Omega 6 and Omega 3 that have proven health benefits when compared to high saturated fat only oils or partially hydrogenated or fully hydrogenated fats that are used in many standard flavored chewy or gummy confections. In addition, other embodiments provide protein to the composition.

One embodiment of the present invention is a composition comprising: a. a monosaccharide component from about 10% to about 40%, by weight of the composition; b. a disaccharide component from about 10% to about 40%, by weight of the composition; c. a dietary fiber component from about 0.001% to about 80%, by weight of the composition; d. a liquid edible oil component from about 1% to about 30% (most preferably a blend of canola, soybean oil and sunflower oil) by weight; e. from about 0.3% to about 20% by weight of a coconut oil component; f. a vanilla powder component from about 0.3% to about 5%, by weight of the composition; g. optionally an emulsifier from about 0.001% to about 5%, by weight of the composition; h. optionally a fruit juice or vegetable juice or fruit or vegetable juice concentrate or fruit or vegetable puree from citrus fruit juices, orchard fruit juices, berry fruit juices, vine fruit juices, decolorized juices or all vegetable juices or combinations thereof component from about 0.001% to about 25%, by weight of the composition; i. optionally a water component at about 0% or greater, by weight of the composition; and j. optional additional additives selected from a flavor component, a color component, a preservative component, a stabilizing component, an emulsifier component, a thickening component, a gelling component like gelatin or pectin, a supplement component that can include a protein source like whey protein isolate or another protein source suitable for processing in a flavored chewy candy confection, a nutraceutical component, a medicine component or combinations thereof.

A preferred flavored chewy or gummy candy confection composition comprises (all percentages are weight percentages, based on the total weight of the composition):

a. monosaccharide component at 18%

b. disaccharide component at 21%

c. inulin dietary fiber component at 24%

d. liquid edible oil component at 12% (blend of canola, soybean oil and sunflower oil)

e. 3% coconut oil component

f. vanilla powder component of 3%

g. corn starch 6%

h. 8% fruit punch concentrate (apple, grape, cherry, passionfruit)

i. 4% water component

j. ascorbic acid 1%

k. a natural preservative tocopherols at 0.001% by weight of composition

An embodiment of the present invention is a composition comprising: a. a monosaccharide component at 18%, by weight of the composition; b. a disaccharide component at 21%, by weight of the composition; and c. an inulin dietary fiber component at 24% of said composition. Another preferred embodiment further comprises d. an edible oil component at 12% by weight of the composition, in which the oil component is comprised of a blend of canola, soybean oil, and sunflower oil. Another preferred embodiment further comprises e. about 3% by weight of a coconut oil component, by weight of the composition. Another preferred embodiment further comprises f. a vanilla powder component at about 3%, by weight of the composition. Another preferred embodiment further comprises g. a modified food starch at about 6%, by weight of the composition; and h. a fruit punch concentrate (apple, grape, cherry, passionfruit) at about 8%, by weight of the composition. Yet another preferred embodiment further comprises i. a water component at about 4%, by weight of the composition. Another preferred embodiment further comprises j. an ascorbic acid component at about 1%, by weight of the composition. Yet another preferred embodiment further comprises k. a natural preservative tocopherols at about 0.001%, based on the total weight of the composition.

A further embodiment of the present invention comprises adding an additional component to the preferred composition described immediately above, in which the additional component is selected from the group consisting of a flavor component, a color component, a preservative component, a stabilizing component, an emulsifier component, a thickening component that includes hydrocolloids including; gelatin, gellan gum, xanthan gum, pectin, carrageenan, cellulose gum, gum arabic, modified starch and combinations thereof, a supplement component that can include organic food acids selected from the group consisting of citric acid, malic acid, lactic acid, fumaric acid, tartaric acid and adipic acid, a nutraceutical component, a medicine component, and combinations thereof.

Another feature of an embodiment of the present invention is a method of making a chewy or gummy confection composition comprising: mixing corn syrup, sugar, inulin, edible oil blend (canola, soybean and sunflower), coconut oil, vanilla powder, corn starch, fruit punch concentrate (apple, grape, cherry, passion fruit), and a water component in a heating vessel; heating the ingredients to a temperature within the range of from about 140 to about 180° F. (60 to 82.2° C.), more preferably within the range of from about 150 to about 160° F. (65.6 to 71.1° C.) to dissolve the monosaccharide and disaccharide components and emulsify the liquid edible oil and coconut oil component to form a uniform heated mixture while scraping the vessel during heating to prevent scorching; continue heating while stirring the ingredients for about 5 minutes to about 30 minutes until the mixture is completely homogeneous; then cooling the mixture to a temperature of about 100° F. (37.8° C.) and blending in the additives such as tocopherols, ascorbic acid and additional flavors, colors, or preservatives while blending with agitation for another 5 minutes or more. Powder food acids also may be added to the finished composition to make the product sour or sugar crystals can be added to make the composition sweet. Confectioners glaze can be added to compositions that will be made into jelly bean shapes like Jelly Bellies. Additional processing also could be carried out on the chewy or gummy confection, such as candy coatings or other confection coatings that are known to be adhered to chewy or gummy confection candies.

While the above example creates a great tasting flavored chewy confection, the preferred embodiments can have a variety of natural or artificial flavors or variations in the additional components within the composition that can produce varieties of flavored chewy or gummy confections similar to the following forms; licorice, taffy, chews, gummies, jelly beans, chewy shapes like bears or other shapes, etc. Depending on the exact level of ingredients used and the flavor profile used, the present embodiments can produce any type of flavored chewy or gummy confection similar to what is available in the market place today.

In another embodiment of the present invention, a flavored chewy or gummy candy confection composition is created using an existing flavored chewy or gummy confection as the base and is modified using the embodiments described herein. The new composition has great taste, texture, mouth feel and has beneficial dietary fiber and can have a balance of dietary fats plus beneficial essential fatty acids Omega 6 and Omega 3 ALA's, and antioxidants.

Such a composition preferably comprises:

a. Flavored chewy or gummy confection candy at 61%

b. Fiber component at 20%

c. Water component at 10% to 20%

d. liquid edible oil component at 0% to 5% (blend of canola, soybean oil and sunflower oil)

e. 0% to 1% coconut oil component

f. vanilla powder component of 0% to 1%

g. emulsifier component at 0% to 2%

h. a natural or artificial flavor component preferably the flavor used in the flavored chewy confection candy in component a. above if necessary

i. natural preservative tocopherols at 0.001% of component

j. 0% to 2% or greater gelatin or another hydrocolloid ingredient to maintain similar gummy chewiness if flavored chewy or gummy confection above is a flavored chewy gummy confection

An embodiment of the present invention is a composition comprising: a. a flavored chewy or gummy confection candy at 61%, by weight of the composition; b. a fiber component at 20%, by weight of the composition; and c. a purified water component from about 10% to about 20%, by weight of the composition. Another embodiment further comprises d. a liquid edible oil component from about 0% to about 5%, by weight of the composition. Another embodiment further comprises e. a coconut oil component present in an amount of from about 0% to about 1%, by weight of the composition. Yet another embodiment further comprises f. a vanilla powder component present in an amount of from about 0% to about 1%, by weight of the composition. Another embodiment further comprises g. an emulsifier component present in an amount of from about 0% to about 2%, by weight of the composition. Another embodiment further comprises h. a natural or artificial flavor component preferably the flavor used in the flavored chewy confection candy in component to provide a flavor profile similar to the original flavored chewy or gummy confection. Yet another embodiment further comprises i. about 0.001% of tocopherols by weight of the finished flavored chewy or gummy candy confection. Another embodiment further comprises j. from about 0% to about 2% or greater gelatin or another hydrocolloid ingredient to maintain similar gummy chewiness, if the flavored chewy or gummy confection is a flavored chewy gummy confection.

Another feature of an embodiment of the present invention is a method of modifying a flavored chewy or gummy confection candy comprising: mixing the flavored chewy or gummy confection candy, a fiber component, a water component, optionally a liquid edible oil component, optionally a coconut oil component, optionally a vanilla powder component, optionally an emulsifier component, optionally a natural or artificial flavor component that preferably is the same or similar to the flavor of the chewy or gummy confection, and optionally a hydrocolloid component like gelatin if the composition is a flavored gummy confection in a heating vessel; heating the ingredients to a temperature within the range of from about 140 to about 180° F. (60 to 82.2° C.), more preferably within the range of from about 150 to about 160° F. (65.6 to 71.1° C.) to melt the flavored chewy or gummy confection, dissolve the sugars, and emulsify the fats and additional ingredients while stirring constantly to prevent scorching for 5 minutes to 30 minutes to make sure the composition becomes completely homogeneous and emulsified; then cooling the mixture to a temperature of about 100° F. (37.8° C.) and adding the 0.001% tocopherols as a natural preservative.

To improve the economics of the above flavored chewy or gummy confection manufacturing process, all the above ingredients including the additional components of a flavor, a color, a preservative, a stabilizing agent, a thickening agent, a supplement, a nutraceutical, a medicine or combinations thereof could be processed together. This would remove the necessity of the second blending and heating stage and would lower the cost of production. Some of the flavored chewy or gummy confection products used as the base for the modified healthier confection structure should be produced during the initial manufacturing process because of colors within the products, powder food acids used to make the products sour and that coat the products, confectioner's glaze used to make the chewy confections glossy like jelly beans, and coatings on the outside of the flavored chewy or gummy confections like Skittles and other coated products like products that have sugar crystals.

Both of the above processes produce a flavored chewy or gummy confection that has great taste, texture, melt, and mouth feel when compared to regular flavored chewy or gummy confections, and that has a balance of dietary fats plus beneficial essential fatty acids Omega 6 and Omega 3 ALA's, antioxidants, lower sugar content, beneficial dietary fiber and other health benefits.

The composition of the preferred embodiments includes a monosaccharide component in an amount of from about 10% to 40%, alternatively 10% to 15%, alternatively 15% to 20%, alternatively 20% to 25%, alternatively 25% to 30%, alternatively 35% to 40%, by weight of the composition. Any monosaccharide can be used in the embodiments, although corn syrup is the preferred monosaccharide. Other suitable monosaccharides that could be used include glucose (dextrose), fructose (levulose), galactose, xylose and ribose. Some sugars within juice or vegetable concentrate or juice or fruit or vegetable source sugar ingredient components also can contribute monosaccharides to the compositions. For example, at least a portion of the monosaccharide can be provided by a fruit or vegetable source selected from the group consisting of citrus fruit juices, orchard fruit juices, berry fruit juices, vine fruit juices, decolorized juices and deflavorized juices in solids provided by its juice, fruit juice powders, fruit purees, fruit nectars, fruit pulp, concentrate fruit juice, and mixtures thereof or vegetable juice, vegetable juice powders, vegetable purees, vegetable pulp, concentrate vegetable juice, and mixtures thereof or mixtures of the above fruit and vegetable sources and dextrose or and any suitable disaccharide including lactose and maltose.

The composition of the preferred embodiments also includes a disaccharide component in an amount of from about 10% to about 40%, alternatively 10% to 15%, alternatively 15% to 20%, alternatively 20% to 25%, alternatively 25% to 30%, alternatively 35% to 40%, by weight of the composition. Any disaccharide may be used in the embodiments, although sucrose or sugar is the preferred disaccharide. Some of the disaccharide may be derived from the optional vanilla powder that is sprayed onto a dextrose base. Other acceptable disaccharide ingredients known to be effective within flavored or chewy confections as sweeteners can be utilized in the embodiments, and the invention is not limited to any particular disaccharide component. In some instances, the monosaccharide and/or disaccharide component may be replaced in whole or in part by a polyol selected from the group consisting of erythritol, sorbitol, maltitol, isomalt, mannitol, xylitol and combinations thereof, or other suitable carbohydrate sweetener acceptable for confectionery use.

The composition of the preferred embodiments also includes a fiber component present in an amount of from about 0.001% to 80%, alternatively 0.001% to 5%, alternatively 5% to 10%, alternatively 10% to 15%, alternatively 15% to 40%, alternatively 40% to 60%, alternatively 60% to 80%, by weight of said composition. Any suitable fiber can be used in the invention, and more preferably a naturally derived fiber is used, including one or more selected from naturally derived inulin, inulin extract, synthetic inulin, hydrolysis products of inulin commonly known as fructooligosaccharides, galacto-oligosaccharides, xylooligosaccharides, oligo derivatives of starch, husks, brans, psyllium, polysaccharides, starches, polycarbophil, lignin, arabinogalactans, chitosans, oat fiber, soluble corn fiber, non-digestible corn dextrin, non-digestible wheat dextrin, locust bean gum and derivatives of locust bean gum, hydroxypropylmethyl cellulose (HPMC), pectin, and mixtures thereof.

The preferred fiber component for use in the preferred embodiments can be selected from inulin, wheat dextrin, or fructooligiosaccharides. Inulin, wheat dextrin, and fructooligiosaccharides act as a thickening agent and also improve the texture of the flavored chewy or gummy confection and add beneficial dietary fiber to the composition. Various load rates of dietary fiber can be incorporated in the composition to create improved texture and at certain load rates can provide dietary benefits including promoting a healthy digestion system, controlling blood sugar levels, and providing probiotic benefits. The addition of the dietary fiber along with the remaining components allow for the addition of water that helps displace sugar within the flavored chewy or gummy confection.

Inulin is indigestible by human enzymes ptyalin and amylase, which are designed to digest starch. As a result, inulin passes through much of the digestive system intact. Inulin is a highly effective prebiotic, stimulating the growth of beneficial probiotic bacteria in the gut. Inulin is used in low fat products because of its ability to provide a creamy smooth texture to products. Inulin is a dietary fiber and is believed to activate beneficial good bacteria in the digestive tract. The activation of these bacteria is thought to reduce the risk of bowel cancer. Inulin has a mildly sweet taste, but does not affect blood sugar levels and is recommended for diabetics. Inulin has been clinically proven to increase calcium absorption. The inherent calcium in dairy foods is now an even better source of this bone-building mineral when inulin is added because inulin improves the body's uptake. People have used plants containing inulin to help relieve diabetes mellitus, a condition characterized by hyperglycemia and/or hyperinsulinemia. The embodiments can provide flavored chewy or gummy candy with inulin as the fiber component and calcium supplementation. This flavored chewy or gummy candy would have an improved absorption of calcium because of the inulin within its composition. Calcium is one of the top deficient minerals in the diet of most individuals, and products produced in accordance with the guidelines provided herein can provide great tasting flavored chewy or gummy confections that can help consumers get more calcium within their diets by eating our products.

The composition of the preferred embodiments also can include edible oil component present in an amount of from about 1% to about 30%, alternatively 1% to 5%, alternatively 5% to 10%, alternatively 10% to 15%, alternatively 15% to 20%, alternatively 20% to 25%, alternatively 25% to 30%, by weight of the composition. This edible oil component makes up part of the group of ingredients that adjust the taste, texture, and improve the melt and mouth feel of the flavored chewy or gummy confection. The interaction of the group of highly unsaturated oils with the coconut oil component creates an improved elasticity within the flavored chewy confection that acts similar to hydrogenated or partially hydrogenated fat. The edible oil component also improves the health characteristic of the flavored chewy confection compositions because it adds monounsaturated and polyunsaturated fats. An example of an edible oil component is a blend of canola, soybean oil, and sunflower oil.

Non-limiting examples of edible oil components acceptable for use in the preferred embodiments include those that have low saturated fat content and high unsaturated fat including monounsaturated and especially polyunsaturated oils. The edible oil component should have no specific flavor and preferably is basically bland or somewhat buttery in taste. The edible oils component can be selected from the following; canola oil, soybean oil, safflower oil, sunflower oil, sesame oil, walnut oil, olive oil, flaxseed oil, chia seed oil, almond oil, corn oil, grape seed oil, peanut oil, other nut oils, and synthesized or reorganized oils, and combinations thereof.

The composition of the preferred embodiments also may include am edible oil component having a high level of saturated fats present in an amount of from about 0.3% to about 20%, alternatively 0.3% to 3%, alternatively 3% to 5%, alternatively 5% to 10%, alternatively 10% to 15%, alternatively 15% to 20%, by weight of the composition. Suitable oils having a high level of saturated fats include, but are not limited to, one or more selected from the group consisting of coconut oil, palm oil, fractionated coconut or palm oil, partially hydrogenated coconut or palm oil, fully hydrogenated coconut or palm oil, or any other synthesized or altered edible oils including partially hydrogenated oils and fully hydrogenated oils that have either highly saturated or highly unsaturated fatty acids that when hydrogenated become solid similar to coconut oil in consistency including partially hydrogenated soybean oil, cotton seed oil, palm kernel oil or combination of these edible oils. Preferred for use in the embodiments is a coconut oil component. This oil component forms a part of the flavor profile and provides a unique texture to the flavored chewy or gummy confection, and it improves the taste, texture, melt, and mouth feel of the compositions. The blend of the flavor components provides for a great taste, texture, melt and mouthfeel, without the necessity of using partially hydrogenated or fully hydrogenated oils. Any medium heat processed coconut oil can be used for the coconut oil component.

The composition of the preferred embodiments also includes a vanilla powder component present in an amount of from about 0.3% to about 5%, alternatively 0.3% to 1%, alternatively 1% to 3%, alternatively 3% to 5%, by weight of the composition. This vanilla powder component comes from a vanilla extract sprayed on a dextrose base. The vanilla powder component may have a specific flavor profile, and Cook's Flavoring Company currently sells a vanilla powder product that has a suitable flavor profile for use in the present invention. Another vanilla powder or natural vanilla extract could be used, but it should have a similar taste profile and should have the same or similar structure as the Cook's Flavoring Company's vanilla powder.

The composition of the preferred embodiments also includes an emulsifier component present in an amount of from about 0.001% to about 5%, alternatively 0.001% to 1%, alternatively 1% to 3%, alternatively 3% to 5%, by weight of the composition. This emulsifier component can come from several different sources. The present invention preferably utilizes modified corn starch, mono-and diglycerides, and lecithin as the primary emulsifier agents. The emulsifier agent acts as an agent to keep the mixture of two or more liquids in which one is dispersed in the other as microscopic or ultramicroscopic droplets. In the flavored chewy or gummy confection of the present invention this is the water and oil emulsion that creates the smooth texture of the finished product.

The composition of the preferred embodiments also may include from about 0.001% to about 25% by weight of a fruit or vegetable or combination juice or concentrate component, alternatively 0.001% to 5%, alternatively 5% to 10%, alternatively 10% to 15%, alternatively 15% to 20%, alternatively 20% to 25%, by weight of the composition. This fruit or vegetable or combination juice or concentrate component adds a flavor to the flavored chewy or gummy confection. Any suitable source from the following may used in the embodiments; citrus fruit juices, orchard fruit juices, berry fruit juices, vine fruit juices, decolorized juices, and vegetable juices can be used for this component. The forms can come from juices or concentrates of fruits or vegetables.

The composition of the preferred embodiments may also include a water component present in an amount of about 0% or greater. The water component may be derived from any suitable purified drinkable water source. The water component adds to the overall texture and melt and chewiness of the flavored chewy or gummy confection. Water is a component that may be used in the preferred embodiments because of the increase in viscosity that the inventive components sometimes may create within the composition. Water is needed to raise the viscosity of the composition to make the texture and taste correct.

Additional embodiments of the flavored chewy or gummy confection compositions can include at least about 0% or greater, by weight of the composition, of an additive selected from a flavor component (natural or artificial), a color component, a preservative component, a stabilizing component, an emulsifier component, a thickening component (e.g., hydrocolloids including gelatin, gellan gum, xanthum gum, pectin, carrageenan, cellulose gum, gum Arabic, modified starch wheat flour or enriched wheat flour or bleached flour or any type of flour from a natural source and combinations thereof), a supplement component that can include a protein source like whey protein isolate or another protein source suitable for processing in a flavored chewy candy or gummy confection, or a supplement component that can include organic food acids selected from the group consisting of citric acid, malic acid, lactic acid, fumaric acid, tartaric acid, adipic acid, and mixtures thereof, a nutraceutical component, a medicine component or combinations thereof.

Non-limiting examples of such other components include: calcium, potassium, B vitamins, vitamins A, C, D, E, and K, folic acid, other vitamins and minerals commonly known in the art and used for supplementing the diet; extracts and active phytochemicals including ferulic acid (from apples), ginseng, ginko biloba, beta carotene, capsicanoids, anthocyanidins, bioflavinoids, d-limonene, isothiocyanates, cysteines from garlic, ginger, grapes, catechins and polyphenols from teas, onions, phytosterols, isoflavones, lycopene, curcumin, caffeine; glucosamine, chondroitin, msm; melatonin, seratonin; and mixtures thereof.

Non-limiting examples of suitable preservatives include: sodium benzoate, sodium citrate, sodium phosphate, potassium metabisulfite, sodium metabisulfite, sodium lactate, sodium sulfite, EDTA (ethylenediaminetetraacetic acid), methylparaben, TBHQ, tocopherols, and mixtures thereof.

The embodiments also include a method of making a chewy or gummy confection comprising: mixing the monosaccharide, disaccharide, a fiber component, a liquid edible oil component, a coconut oil component, a vanilla powder component, an emulsifier component, a fruit or vegetable juice or concentrate or combinations of fruit and vegetables, and a water component in a heating vessel; heating the ingredients to a temperature within the range of from about 140 to about 180° F. (60 to 82.2° C.), more preferably within the range of from about 150 to about 160° F. (65.6 to 71.1° C.) to dissolve the monosaccharide and disaccharide components and emulsify the liquid edible oil and coconut oil component to form a uniform heated mixture while scraping the vessel during heating to prevent scorching; continue heating while stirring the ingredients for about 5 minutes to about 30 minutes until the mixture is completely homogeneous; then cooling the mixture to a temperature of about 100° F. (37.8° C.) and about 0.001% tocopherols as a natural preservative. An additional color component, a preservative component, a stabilizing component, an emulsifier component, a thickening component, a gelling component like gelatin or pectin, a supplement component that can include a protein source like whey protein isolate or another protein source suitable for processing in a flavored chewy candy or gummy confection, a nutraceutical component, a medicine component or combinations thereof can be added to the flavored chewy or gummy confection. Some of the above additional components should be added at the original mixing point like a protein source or gelling component like gelatin. If the component needs to be mixed under heat then it should be added in the mixing stage, or if it can be blended into the composition after the mixing stage then it can be added at the cooling stage. Powder food acids or confectioner's glaze can be added or other candied coatings like that present on the Skittles product can be added to the chewy or gummy confection depending on the format of product being produced.

The method further includes a final processing process in which the final product is prepared. This process can include, for example, extruding, thermoforming, molding, shaping, cutting, and the like, to form the final product in the desired shape. Those skilled in the art are capable of designing a suitable final processing procedure to prepare the final chewy and gummy candy confection, depending on the desired texture (e.g., chewy or gummy) and shape (e.g., cube, square, sheet, animal shaped, etc.), using the guidelines provided herein.

An additional embodiment of the present invention includes a modified flavored chewy or gummy confection candy comprising: a. a flavored chewy or gummy confection candy present in an amount of from about 30% to about 90%, by weight of the composition; b. a fiber component present in an amount of from about 0.001% to about 60%, by weight of the composition; c. a water component present in an amount of from about 1% to about 20%, by weight of the composition; d. a liquid edible oil component present in an amount of from about 0% to about 30%, by weight of the composition; e. a coconut oil component present in an amount of from about 0% to about 20%, by weight of the composition; f. a vanilla powder component present in an amount of from about 0% to about 5%, by weight of the composition; g. an emulsifier component present in an amount of from about 0% to about 5%, by weight of the composition; h. a natural or artificial flavor component that preferably has the same or similar flavor used in the flavored chewy confection candy to provide a flavor profile similar to the original flavored chewy or gummy confection used as component (a); i. a 0.001% tocopherols present in an amount of about 0.001% by weight of finished flavored chewy or gummy candy confection; and j. from about 0% to about 2% or greater gelatin or another hydrocolloid ingredient to maintain similar gummy chewiness if flavored chewy or gummy confection above is a flavored chewy gummy confection.

The composition of the present invention comprises an existing flavored chewy or gummy confection candy at 30% to 90%, alternatively 30% to 40%, alternatively 40% to 50%, alternatively 50% to 60%, alternatively 60% to 70%, alternatively 70% to 80%, alternatively 80% to 90%, by weight of said composition. This flavored chewy or gummy candy confection base can be any product as long as it has a monosaccharide and disaccharide component. Exemplary chewy or gummy candy confections include, but are not limited to, one or more of the following, Twizzlers, Red Vines, Licorice, Airheads, Laffy Taffy, Starbursts, Swedish Fish, Skittles, Tootsie Roll, Gummy Bears, Lifesavors Gummy Candies, Welch's fruit snacks, Sour Patch Kids or any other flavored chewy or gummy candy confection that has a monosaccharide and a disaccharide component.

In some cases, the flavored chewy or gummy confection used as the base may have to be adjusted in the original processing stage so that the final coatings or flavor crystals or colors within the flavored chewy or gummy confection can be maintained. In this case the adjustment of the base flavored or gummy confection used within the composition would happen at the original processing of the product. It may also be advantageous to process all the ingredients together during the original production of the base chewy or gummy confection to lower the cost and remove the necessity for double processing. Using the guidelines provided herein, those having ordinary skill in the art will be capable of preserving the final coating or flavor crystals or colors of the original flavored chewy or gummy confection.

The composition of this embodiment also preferably a fiber component present in an amount of from about 0.001% to about 60%, alternatively 0.001% to 5%, alternatively 5% to 15%, alternatively 15% to 25%, alternatively 25% to 40%, alternatively 40% to 60%, by weight of the composition. The same fiber components discussed above can be used in this embodiment. The preferred fiber component for use in the preferred embodiments can be selected from inulin, wheat dextrin, or fructooligiosaccharides. Inulin, wheat dextrin, and fructooligiosaccharides act as a thickening agent and also improve the texture of the flavored chewy or gummy confection and add beneficial dietary fiber to the composition. Various load rates of dietary fiber can be incorporated in the composition to create improved texture and at certain load rates can provide dietary benefits including promoting a healthy digestion system, controlling blood sugar levels, and providing probiotic benefits. The addition of the dietary fiber along with the remaining components allow for the addition of water that helps displace sugar within the flavored chewy or gummy confection.

The composition of these embodiments also preferably a water component present in an amount of from about 1% to about 20%, alternatively 1% to 5%, alternatively 5% to 10%, alternatively 10% to 15%, alternatively 15% to 20%, by weight of the composition. This water component may come from any suitable purified drinkable water source. The water component adds to the overall texture, melt and chewiness of the flavored chewy or gummy confection. Water is a component within the embodiments because of the increase in viscosity that the ingredients may create within the composition. In order to make the final composition similar to the original flavored chewy or gummy confection, water may be needed to decrease the viscosity of the composition to make the texture and taste similar to that of the original product prior to the inventive modification. A feature of the invention is prepare the final flavored chewy or gummy confection to have as similar a viscosity to the original product, and consequently, water may sometimes be added. For some flavored chewy or gummy confections that can be altered, the fiber component and water component are the only two ingredients necessary to improve the health characteristics and add beneficial dietary fiber to the final flavored chewy or gummy confection.

Other preferred embodiments for modifying existing flavored chewy or gummy confection candy products include the addition of further ingredients, such as an edible oil component. The edible oil component may be present in an amount of from about 0% to about 30%, alternatively 0% to 1%, alternatively 1% to 5%, alternatively 5% to 10%, alternatively 10% to 15%, alternatively 15% to 20%, alternatively 20% to 25%, alternatively 25% to 30%, by weight of the composition. The edible oil component may include the edible oil having a high level of unsaturated fats discussed in the embodiments above, as well as the edible oil having a high level of saturated fats discussed in the embodiment above, and mixtures thereof. This edible oil component makes up part of the group of ingredients that adjust the taste, texture, and mouth feel of the flavored chewy or gummy candy confection. The edible oil with high levels of unsaturated fat may interact with the optional coconut oil component and this creates a unique new improved elasticity. The edible oil component also improves the health characteristic of the flavored chewy or gummy confection compositions because it adds monounsaturated and polyunsaturated fats. An example of a preferred edible oil component is a blend of canola, soybean oil, and sunflower oil. Non-limiting examples of an edible oil component acceptable for use in the preferred embodiments are those that should have low saturated fat content and high unsaturated fat including monounsaturated and especially polyunsaturated oils. The edible oil component should have no specific flavor and preferably is essentially bland or somewhat buttery in taste. The edible oils component can come from the following; canola oil, soybean oil, safflower oil, sunflower oil, sesame oil, walnut oil, olive oil, flaxseed oil, chia seed oil, almond oil, corn oil, grape seed oil, peanut oil, other nut oils, and synthesized or reorganized oils, and combinations thereof.

The composition of the preferred embodiments also may optionally include a coconut oil component in an amount of from about 0% to about 20%, 0% to 1%, alternatively 1% to 3%, alternatively 3% to 5%, alternatively 5% to 10%, alternatively 10% to 15%, alternatively 15% to 20%, by weight of said composition. It is preferred that the coconut oil component has a mild flavor profile and not to have an overpowering flavor profile. The preferred oil for use in the preferred embodiments is a refined medium heat coconut oil. Other forms of coconut oil could be used, but it would be important that the taste does not alter the taste of the base flavored chewy or gummy confection being adjusted.

The composition of the preferred embodiments also may include a vanilla powder component present in an amount of from about 0% to about 5%, alternatively 0.3% to 1%, alternatively 1% to 3%, alternatively 3% to 5%, by weight. This vanilla powder component preferably comes from a vanilla extract sprayed on a dextrose base. The blend of flavor components is believed to assist in accomplishing the comparable taste, texture, melt and mouth feel without the necessity for adding partially hydrogenated or fully hydrogenated fats. Therefore, the vanilla powder component should have a specific flavor profile and Cook's Flavoring Company currently sells a vanilla powder that has a suitable flavor profile for use in the present invention. Another vanilla powder or natural vanilla extract could be used, but it would have to have the same or similar taste profile and should have the same structure as the Cook's Flavoring Company's vanilla powder.

The composition of the preferred embodiments also may include an emulsifier component present in an amount of from about 0% to about 5%, alternatively 0.001% to 1%, alternatively 1% to 3%, alternatively 3% to 5%, by weight of an emulsifier component. The emulsifier component is believed to assist in holding together the fats and water and other components together in a homogeneous composition. The preferred emulsifier is corn starch, mono- and diglycerides, lecithin or combinations thereof, depending on the type of flavored chewy or gummy confection being used as the base for the composition. Other emulsifiers can be used that are known to be effective within flavored chewy or gummy confections.

Additional embodiments of the flavored chewy or gummy confection compositions can include at least about 0% or greater, by weight of the composition of an additive or supplement component including, but not limited to, vitamins, minerals, herbs, botanicals, plant derived supplements, protein supplements, animal derived supplements, therapeutic compounds, and mixtures thereof. Suitable additives include any of the additives or supplements discussed in the embodiments above.

Non-limiting examples of other components that may be added to modify the chewy or gummy candy confection include, but are not limited to one or more of the following: calcium, potassium, B vitamins, vitamins A, C, D, E, and K, folic acid, other vitamins and minerals commonly known in the art and used for supplementing the diet; extracts and active phytochemicals including ferulic acid (from apples), ginseng, ginko biloba, beta carotene, capsicanoids, anthocyanidins, bioflavinoids, d-limonene, isothiocyanates, cysteines from garlic, ginger, grapes, catechins and polyphenols from teas, onions, phytosterols, isoflavones, lycopene, curcumin, caffeine; glucosamine, chondroitin, msm; melatonin, seratonin; and mixtures thereof.

Alternatively, the composition of the present invention can include from about 0% to about 25%, alternatively from about 0% to about 10%, and alternatively from about 0% to about 5%, by weight of the composition, of a supplement component.

Non-limiting examples of suitable preservatives include: sodium benzoate, sodium citrate, sodium phosphate, potassium metabisulfite, sodium metabisulfite, sodium lactate, sodium sulfite, EDTA (ethylenediaminetetraacetic acid), methylparaben, TBHQ, tocopherols, and mixtures thereof. The compositions of the present invention can include at least about 0% to 2%, by weight of the composition of a preservative component from above, or mixtures thereof.

Another feature of an embodiment of the present invention is a method of modifying a flavored chewy or gummy confection candy comprising: mixing the flavored chewy or gummy confection candy, a fiber component, a water component, optionally a liquid edible oil component, optionally a coconut oil component, optionally a vanilla powder component, optionally an emulsifier component, optionally a natural or artificial flavor component that preferably is the same or similar to the flavor of the chewy or gummy confection, and optionally a hydrocolloid component like gelatin if the composition is a flavored gummy confection in a heating vessel; heating the ingredients to a temperature within the range of from about 140 to about 180° F. (60 to 82.2° C.), more preferably within the range of from about 150 to about 160° F. (65.6 to 71.1° C.) to melt the flavored chewy or gummy confection, dissolve the sugars, and emulsify the fats and additional ingredients while stirring constantly to prevent scorching for 5 minutes to 30 minutes to make sure the composition becomes completely homogeneous and emulsified; then cooling the mixture to a suitable temperature, preferably a temperature of about 100° F. (37.8° C.) and adding the 0.001% tocopherols as a natural preservative.

An additional color component, flavor component, a preservative component, a stabilizing component, an emulsifier component, a thickening component, a gelling component like gelatin or pectin, a supplement component that can include a protein source like whey protein isolate or another protein source suitable for processing in a flavored chewy candy or gummy confection, a nutraceutical component, a medicine component or combinations thereof can be added to the flavored chewy or gummy confection. Some of the above additional components should be added at the original mixing point like a protein source or gelling component like gelatin. If the component is mixed under heat then it would be added in the mixing stage or if it can be blended into the composition after the mixing stage then it would be added at around the cooling stage. Powder food acids or confectioner's glaze can be added or other coatings like on Skittles can be added to the chewy or gummy confection depending on the format of product being produced.

The method further includes a final processing process in which the final product is prepared. This process can include, for example, extruding, thermoforming, molding, shaping, cutting, and the like, to form the final product in the desired shape. Those skilled in the art are capable of designing a suitable final processing procedure to prepare the final chewy and gummy candy confection, depending on the desired texture (e.g., chewy or gummy) and shape (e.g., cube, square, sheet, animal shaped, etc.), using the guidelines provided herein.

To improve the economics of the above flavored chewy or gummy confection the manufacturing process for all the above ingredients including the additional component of a flavor, a color, a preservative, a stabilizing agent, a thickening agent, a supplement or combinations thereof could be processed together. This would remove the necessity of the second blending and heating stage and would lower the cost of production.

The preferred embodiments now will be described in more detail with reference to the non-limiting examples that follow.

### EXAMPLES

### Example 1

A cranberry flavored chewy confection with improved health characteristics, lower sugar content, balance of dietary fats, beneficial antioxidants and dietary fiber.

Approximately 15 pounds of corn syrup, 18 pounds of sugar, 12 pounds of Crisco Vegetable Oil Blend (Canola, Soybean, and Sunflower), 3 pounds of coconut oil medium refined, 3 pounds of vanilla powder supplied by Cooks Flavor Company, 18 pounds of inulin, 3 pounds of Wheat Dextrin, 5 pounds water, 1 pound of Lecithin, 1 pound of modified corn starch, and 7 pounds of cranberry juice concentrate were added and mixed in a kettle equipped with a gas heater and mixer. The ingredients were mixed together and warmed to 150 to 160° F. over a low flame, while continuously scraping the side of the kettle to prevent scorching for about 10 minutes to dissolve the sugar and emulsify the fat, while maintaining the temperature at 150 to 160° F. The batch then was tested for thickness and was either taken off the flame and cooled or slowly boiled for an additional period of time to create the required viscosity for the flavored chewy confection being chosen to be made. The batch then was cooled to about 100° F., and about 0.001% of tocopherols were added the batch and blended into the mixture. The resulting product was a cranberry flavored chewy confection.

### Example 2

A pineapple/orange flavored gummy confection with improved health characteristics, balance of dietary fats, beneficial antioxidants and dietary fiber.

About 15 pounds of corn syrup, 10 pounds of sugar, 5 pounds of Crisco Vegetable Oil Blend (Canola, Soybean, and Sunflower), 1 pound of coconut oil medium refined, 1 pound of vanilla powder supplied by Cooks Flavor Company, 13 pounds of inulin, 3 pounds of Wheat Dextrin, 5 pounds water, 1 pound of Lecithin, 5 pounds of modified corn starch, 15 pounds of pineapple/orange juice concentrate, and 10 pounds of gelatin were added and mixed together in a kettle equipped with a gas heater and mixer. The mixture was heated to 150 to 160° F. over a low flame, while continuously scraping the side of the kettle to prevent scorching for about 10 minutes to dissolve the sugar and emulsify the fat. The composition was then cooled to 100° F., and 0.001% of tocopherols as a natural preservative were added to the composition. This type of process could be extrapolated into large scale production. The final product was a pineapple/orange flavored gummy confection.

### Example 3

Orange Starburst chews having improved health characteristics including; antioxidant content, reduced sugar content, beneficial dietary fiber and dietary benefits, when compared to the original orange Starburst.

About 60 pounds of Starburst orange flavored chew confection, 20 pounds of inulin, 4 pounds of Crisco Vegetable Oil Blend (Canola, Soybean, and Sunflower), 1.5 pounds of coconut oil refined medium heat, 1.5 pounds of vanilla powder supplied by Cooks Flavor Company, 3 pounds of corn starch, and 10 pounds of water were added and mixed together in a kettle equipped with a gas heater and mixer. The ingredients were mixed together and warmed to 150 to 160° F. over a low flame while continuously scraping the side of the kettle to prevent scorching for about 10 minutes to dissolve the vanilla powder on the dextrose base and emulsify the fat. The mixture then was cooled to about 100° F., and tocopherols added at 0.001% based on the total weight of the composition. Additional flavors similar to the orange starburst flavor, as well as additional coloring, were added if needed to bring the taste and color in line with the original product. The product then was processed in an extrusion machine and cut into square pieces to produce an improved orange Starburst.

This above composition for creating an improved health characteristic Starburst orange flavored chew can be scaled up to commercial production. It also may be advantageous to create the composition all at once in order to save production costs.

### Example 4

Welch's fruit with improved health characteristic including antioxidant content, reduced sugar content, beneficial dietary fiber and dietary benefits, when compared to the original Welch's fruit.

About 65 pounds of Welch's fruit snacks grape flavored gummy confection, 12 pounds of inulin, 4 pounds of Crisco Vegetable Oil Blend (Canola, Soybean, and Sunflower), 1.5 pounds of coconut oil refined medium heat, 1.5 pounds of vanilla powder supplied by Cooks Flavor Company, 3 pounds of corn starch, 6 pounds of water, and 7 pounds of whey protein isolate were added and mixed together in a kettle equipped with a gas heater and mixer. The ingredients were mixed together and warmed to 150 to 160° F. over a low flame while continuously scraping the side of the kettle to prevent scorching for about 10 minutes to dissolve the vanilla powder on the dextrose base and emulsify the fat. The mixture then was cooled to about 100° F., and tocopherols added at 0.001% based on the total weight of the composition. Additional flavors similar to the original Welch's fruit snacks grape flavored gummy, as well as additional coloring were added if needed to bring the taste and color in line with the original product. The product then was processed in a flavored gummy confection processing line and molded into the fruit shapes.

This above composition for creating an improved health characteristic Welch's grape flavored gummy can be scaled up to commercial production. It also may be advantageous to create the composition all at once in order to save production costs.

The above two examples produced great tasting flavored chewy or gummy confections. Any flavored chewy or gummy confection that has a monosaccharide and disaccharide component can be used as the base for this embodiment. The embodiments can provide any suitable style of flavored chewy or gummy confections known to be available in the industry.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition comprising: (a) a monosaccharide component present in an amount of from about 10% to about 40% by weight, based on the total weight of the composition; (b) a disaccharide component present in an amount of from about 10% to about 40% by weight, based on the total weight of the composition; and (c) a dietary fiber component present in an amount of from about 0.001% to about 80% by weight, based on the total weight of the composition.

2. The composition as claimed in claim 1, further comprising: (d) an edible oil component that has a highly unsaturated fat component present in an amount of from about 1% to about 30%, by weight of the composition.

3. The composition as claimed in claim 2, further comprising: (e) an edible oil component that has a highly saturated fat content component present in an amount of from about 0.3% to about 20%, by weight of the composition.

4. The composition as claimed in claim 3, further comprising: (f) a vanilla powder component present in an amount of from about 0.3% to about 5%, by weight of the composition.

5. The composition as claimed in claim 4, further comprising: (g) an emulsifier component present in an amount of from about 0.001% to about 5%, by weight of the composition.

6. The composition as claimed in claim 5, further comprising (h) a juice selected from the group consisting of fruit juice, vegetable juice, fruit juice concentrate, vegetable juice concentrate, fruit puree, vegetable puree, and mixtures thereof, present in an amount of from about 0.001% to about 25%, by weight of the composition.

7. The composition as claimed in claim 6, further optionally comprising (i) water.

8. The composition as claimed in claim 7, further comprising an additive selected from the group consisting of a flavor component, a color component, a preservative component, a stabilizing component, an emulsifier component, a thickening component, a gelling component, a protein source, a nutraceutical component, a medicine component, and mixtures thereof.

9. A modified flavored chewy or gummy confection candy comprising: (a) a flavored chewy or gummy confection candy present in an amount of from about 30% to about 90% by weight, based on the total weight of the composition; (b) a fiber component present in an amount of from about 0.001 to about 60% by weight of the composition; and (c) a water component from about 1% to 20%, by weight of the composition composition.

10. The modified flavored chewy or gummy confection candy as claimed in claim 9, further comprising: (d) an edible oil component that has a highly unsaturated fat component present in an amount of from about 0% to about 30%, by weight of the composition; and (e) an edible oil component that has a highly saturated fat content component present in an amount of from about 0% to about 20%, by weight of the composition.

11. The modified flavored chewy or gummy confection candy as claimed in claim 10, further comprising: (f) a vanilla powder component present in an amount of from about 0% to about 5%, by weight of the composition.

12. The modified flavored chewy or gummy confection candy as claimed in claim 11, further comprising: (g) an emulsifier component present in an amount of from about 0% to about 5%, by weight of the composition

13. The modified flavored chewy or gummy confection candy as claimed in claim 9, further comprising one or more additives selected from the group consisting of a flavor component, a color component, a preservative component, a stabilizing component, an emulsifier component, a thickening component, a gelling component, a protein source, a nutraceutical component, a medicine component, or combinations thereof.

14. A method of making the composition of claim 1 comprising: (a) mixing at least the monosaccharide component, the disaccharide component, and the dietary fiber component in a vessel; (b) heating the mixture, while mixing and scraping, to a temperature within the range of from about 60 to about 82.2° C. for a period of time of from about 5 minutes to about 30 minutes to provide a homogeneous mixture; (c) optionally cooling the mixture to a temperature of about 37.8° C. and adding a natural preservative; (d) processing the mixture to form a chewy or gummy candy confection.

15. The method of claim 14, further comprising adding to the mixture of process (a) one or more of the following ingredients selected from the group consisting of an edible oil having a highly unsaturated fat component, an edible oil having a highly saturated fat component, a vanilla powder component, an emulsifier component, a juice, water, and mixtures thereof.

16. The method of claim 14, further comprising adding to the homogeneous mixture of (c) one or more additives selected from the group consisting of a flavor component, a color component, a preservative component, a stabilizing component, an emulsifier component, a thickening component, a gelling component, a protein source, a nutraceutical component, a medicine component, and mixtures thereof.

17. A method of making the modified chewy or gummy candy confection as claimed in claim 9 comprising: (a) mixing at least the chewy or gummy candy confection, the dietary fiber component, and the water component in a vessel; (b) heating the mixture, while mixing and scraping, to a temperature within the range of from about 60 to about 82.2° C. for a period of time of from about 5 minutes to about 30 minutes to provide a homogeneous mixture; (c) optionally cooling the mixture to a temperature of about 37.8° C. and adding a natural preservative; (d) optionally adding additional flavors and colors to match the original flavor and color of the chewy or gummy candy confection added in (a); and (e) processing the mixture to form a chewy or gummy candy confection.

18. The method as claimed in claim 17, further comprising mixing in (a) one or more of the following ingredients selected from the group consisting of an edible oil having a highly unsaturated fat component, an edible oil having a highly saturated fat component, a vanilla powder component, an emulsifier component, a juice, and mixtures thereof.

19. The method as claimed in claim 17, further comprising adding to the homogeneous mixture of (d) one or more additives selected from the group consisting of a flavor component, a color component, a preservative component, a stabilizing component, an emulsifier component, a thickening component, a gelling component, a protein source, a nutraceutical component, a medicine component, and mixtures thereof.

20. A composition comprising the following by weight, based on the total weight of the composition:
a. 18% to 23% of a monosaccharide component;
b. 21% to 26% of a disaccharide component;
c. 15% to 24% of a fiber component;
c. 12% to 15% of a liquid edible oil component comprising a blend of canola, soybean oil and sunflower oil;
d. 3% to 5% of a coconut oil component;
e. 3% of a vanilla powder component;
g. 0.001% to 5% of an emulsifier component;
h. 8% of a fruit or vegetable juice concentrate component;
i. 4% of water;
j. a natural or artificial flavor component;
k. a natural or artificial color component; and
l. 0.001% of a natural preservative.
